(19) United States
(12) Patent Application Publication
(10) Pub. No.: US 2026/0167995 A1
(43) Pub. Date: Jun. 18, 2026

(54) PROCESS OF PRODUCING BIOFUEL USING MICROORGANISMS AND CARBON DIOXIDE EXTRACTED FROM INDUSTRIAL CHIMNEYS

(71) Applicant: Majid Hajibarati, Tehran (IR)

(72) Inventor: Majid Hajibarati, Tehran (IR)

(21) Appl. No.: 18/981,814

(22) Filed: Dec. 16, 2024

(51) Int. Cl. C12P 7/6436; B01J 23/04; C01B 32/55; C11C 3/00; C12R 1/01

(52) U.S. Cl. CPC C12P 7/6436; B01J 23/04; C01B 32/55; C11C 3/003; C12R 2001/01

(57) ABSTRACT

The invention of a process of producing biofuel using microorganisms and carbon dioxide extracted from industrial chimneys includes the separation and purification of carbon dioxide utilizing absorption and disposal towers and the use of solvents such as monoethanolamine. The purified CO₂ is then injected into the cyanobacteria cultivation system in shallow pools with insulated walls and a glass or polycarbonate coating. Through aeration and mechanical stirring, photosynthesis and the growth of cyanobacteria, such as the Synechocystis sp. PCC 6803, is maximized. The cyanobacteria biomass is separated using centrifugation, dried, and ground. The lipids present in the biomass are extracted using solvents such as hexane or methanol under controlled conditions. The extracted lipids are then converted into biodiesel in a chemical reactor through a transesterification process with alcohol and a catalyst, while glycerol is collected as a by-product for use in the cosmetic and personal care industries.

### TECHNICAL FIELD OF INVENTION

The invention relates to a process for producing biofuel through the cultivation of cyanobacteria using carbon dioxide recovered from the exhaust gases of industrial chimneys. It also pertains to a process for the separation and purification of carbon dioxide, as well as to a system for cultivating cyanobacteria. Additionally, the invention involves the extraction of lipids from biomass and the production of biodiesel.

### PRIOR ARTS

In recent decades, significant efforts have been made to reduce greenhouse gas emissions and utilize renewable energy sources. Common methods include carbon dioxide capture and storage, as well as biofuel production from biomass. However, current methods for directly converting industrial carbon dioxide into biofuel are inefficient and costly. Biofuels are produced from renewable energy sources such as plants, algae, organic waste, and other biomass materials. These fuels include bioethanol, biodiesel, and biogas.

These biofuels have gained attention due to their potential to reduce reliance on fossil fuels, lower greenhouse gas emissions, and promote sustainable development. Microorganisms such as bacteria, algae, and fungi play a crucial role in the production of these fuels. These organisms can carry out the conversion process at a high rate and under mild environmental conditions.

Carbon dioxide is one of the primary greenhouse gases produced as a result of excessive human activities. Various strategies exist to reduce carbon dioxide emissions, but using it as a raw material in industrial processes, including biofuel production, is of particular importance. Moreover, by converting carbon dioxide into biofuels, its release into the atmosphere can be prevented. The fuels produced can replace fossil fuels and contribute to the overall reduction of greenhouse gases.

Biofuel production from greenhouse gases, especially carbon dioxide (CO₂), using microorganisms is an innovative approach to reducing greenhouse gas emissions and producing sustainable energy. This technology is based on microorganisms such as bacteria, algae, and fungi, which are capable of absorbing CO₂ and converting it into biofuels. Biofuels produced from microorganisms can be used in transportation, industry, and power generation as a clean and renewable energy source. Utilizing high concentrations of carbon dioxide and collecting it from industrial sources or natural environments are crucial steps in producing biofuels from CO₂.

An Australian invention with patent No. AU2011327956B2 which was granted on Nov. 12, 2015 titled “A method for lipid extraction from biomass” relates to a method for recovering lipids from wet microbial biomass, where lipids are extracted without disrupting the cell walls. In this method, wet biomass with a specific dry matter content (less than 45% by weight, typically between 5% and 38%) is contacted with a liquid extractor at high temperatures (ranging from 170° C. to 300° C.) and under high pressure, without breaking the cell walls. The combination of temperature and pressure ensures that the lipids inside the cells come into contact with the extractor and are extracted. The extractor can include non-polar organic solvents such as aliphatic or cyclic alkanes with carbon chain lengths from C₃ to C₁₆ (e.g., hexane, heptane, octane), and may also include polar, water-miscible solvents like alcohols or organic acids. The dry biomass to total extractor ratio is set between 1:1 and 1:20. After extraction, the extracted lipids are recovered from or along with the extractor. This method is applicable to various types of microbial biomass, such as bacteria, cyanobacteria, fungi, algae, and other microorganisms, and allows for the addition of extra water to the biomass before, during, or after the extraction process.

A Chinese invention with publication No. CN113388649A which was filed on Nov. 29, 2013 titled “Fermentation process” relates to the fields of biotechnology and chemical engineering and describes a method for producing at least one lipid product from gaseous substrates such as carbon dioxide (CO₂) and hydrogen (H₂). The method includes the following steps: First, a mixture containing CO and/or H₂ is introduced into a first bioreactor, which contains microorganisms such as Streptobacter or Clostridium in a nutrient-rich liquid medium. Under anaerobic conditions, fermentation takes place, producing acids such as acetic acid or other acids, as well as alcohols like ethanol. Then, a portion of the produced acid is transferred to a second bioreactor, which contains microalgae such as Cladomona, Spirulina, cyanobacteria, or other microalgal species in a nutrient-rich liquid medium. Under aerobic conditions, acid fermentation occurs to produce at least one lipid product. The fermentation culture from the second bioreactor is passed through an oxygen scrubber to remove oxygen, and the purified fermentation culture is returned to part of the first bioreactor. In this process, the acid used may include acetic acid, butyric acid, succinic acid, lactic acid, or propionic acid, and the acetic acid production in the first bioreactor is at least 10 grams per liter per day. The lipid products produced can be used to generate renewable fuels such as renewable diesel, fatty acid methyl esters (FAME), fatty acid ethyl esters (FAEE), and hydrogenated biodiesel.

An European invention with patent No. EP2238231B1 which was granted on Mar. 23, 2016 titled “Transgenic photosynthetic microorganisms and photobioreactor” relates to transgenic cyanobacteria cells that contain an artificial DNA construct. This construct includes a highly efficient cyanobacteria promoter, nucleotides encoding enzymes with disaccharide phosphate synthase and disaccharide phosphatase activities, and a transcription termination sequence. The aim of this genetic engineering is to increase the accumulation of disaccharides, such as sucrose, trehalose, glucosylglycerol, and mannosylfructose, in cyanobacteria compared to non-transgenic cells. These transgenic cells, optimized using various cyanobacteria species such as Synechococcus and Synechocystis, under specific conditions (pH between 4.5 and 6 and temperature between 20 to 30° C.), are designed to enhance disaccharide production. Additionally, some DNA sequences include complementary sequences and structures that enable disaccharide secretion from the cells, improving the production and extraction process of disaccharides. This method, by utilizing inducible promoters and precise environmental control, allows for the production of larger quantities of disaccharides, which can have widespread applications in the production of food, pharmaceuticals, and renewable energy sources.

A Japanese invention with patent No. JP5305532B2 which was granted on Oct. 2, 2013 titled “Excellent diffused light large surface area water-supported photobioreactor” a scalable photobioreactor system for the efficient production of photosynthetic microorganisms such as microalgae and cyanobacteria is described. In various configurations of this system, features may include an expanded surface area to reduce light intensity and increase photosynthetic yield, an external water reservoir to provide structure and thermal regulation at low cost, flexible plastic or composite panels that are connected and arranged in triangular or other shapes when partially submerged in water, and the use of positive gas buoyancy and pressure to maintain the structural integrity of the photobioreactor chambers. Additionally, the system may employ structures designed to optimize the distribution of scattered light. Other configurations involve air tubes made of plastic film at the bottom of each photobioreactor chamber to provide dispersed air bubbles to the chamber. The photobioreactor system design also includes gas exchange, temperature control, air pumping, liquid pumping, filtration, culture medium recycling, and harvesting methods. For biofuel production, the photobioreactor system may incorporate a separate growth photobioreactor and a secondary stress reactor.

A Japanese invention with patent No. JP6444386B2 which was granted on Dec. 26, 2018 titled “Production of fuel and biofertilizer from biomass” relates to a method for producing liquid fertilizer from biomass based on liquid fuel and cyanobacteria, which includes the following steps: First, the direct melting of edible biomass is carried out using hydroprocessing under high-temperature conditions (at least 170° C.) and appropriate pressure to produce a hydrocarbon liquid. Then, the produced hydrocarbon liquid is upgraded, and ammonia is generated as a byproduct for the production of liquid fuel or chemical feedstock. Next, structured biochar and carbon dioxide are produced as a byproduct through microwave pyrolysis of the remaining biomass. The carbon dioxide resulting from the direct melting and biochar production processes is used in a photobioreactor for the cultivation of cyanobacteria. Finally, a biofertilizer is produced by combining biochar and cyanobacteria. This method also includes the addition of diazotrophic microorganisms to enhance fertilizer performance, removal of minerals from biomass or hydrocarbon liquid before direct melting by adsorption into the biochar, and the reuse of the culture medium after filtration and sterilization. The final biofertilizer consists of a mixture of cyanobacteria, diazotrophic microorganisms, and structured biochar with an average pore size between 20 and 400 microns, which helps improve the carbon and active nitrogen content in the soil. This method aims to optimize environmental and economic processes, enabling the production of sustainable and effective biofertilizers.

A Japanese invention with patent No. JP7034907B2 which was granted on Mar. 4, 2022 titled “Biomass breeding methods and systems and treatment plants” relates to a scalable photobioreactor system for the efficient production of photosynthetic microorganisms such as microalgae and cyanobacteria. This system includes a biomass growth module (BGM) that cultivates the biomass and transfers the biomass-containing wastewater to multiple other BGMs. Additionally, there is a thermal power plant module that generates carbon dioxide gas by burning the produced energy, which is used as fuel for biomass growth. The system is designed such that the wastewater output from the BGM is partially or fully treated using heat from the thermal module, with the generated carbon dioxide significantly enhancing the carbon content of the wastewater. Furthermore, the system includes a diverse water supply for the BGMs, along with primary water treatment processes such as screening, sand and gravel removal, sedimentation, and chemical addition to ensure the water is suitable for input into the BGMs. For biofuel production, the system can include a separate growth photobioreactor and a secondary stress reactor. The system design also incorporates gas exchange, temperature control, air and liquid pumping, filtration, culture medium recycling, and harvesting methods. By integrating various modules, such as the thermal power plant, BGM, filtration module, gasification module, and others, the system enables the sustainable production of biofuels, optimizes resource consumption, reduces environmental pollution, and improves economic efficiency.

A US invention with U.S. Pat. No. 7,682,821B2 which was granted on Mar. 23, 2010 titled “Closed photobioreactor system for continued daily in situ production, separation, collection, and removal of ethanol from genetically enhanced photosynthetic organisms” relates to a closed photobioreactor system designed for the efficient production of photosynthetic microorganisms such as microalgae and cyanobacteria. The system includes a chamber composed of several sections: (a) a headspace, (b) the upper section of the chamber, which has a transparent or glass area for sunlight entry, (c) the lower section of the chamber, which contains a growth medium with a culture of genetically enhanced microorganisms that continuously produce ethanol and release it into the growth medium, (d) multiple openings for input and output tubes, and (e) a collection tray inside the upper section of the chamber that collects condensed droplets containing ethanol and water from the inner surface of the chamber. Additionally, the photobioreactor includes output tubes connected to the collection tray for harvesting the condensed ethanol, an output tube for removing O₂, ethanol, and water vapor from the headspace, input tubes for introducing CO₂ and water into the growth medium, and input tubes for adding aqueous solutions containing nutrients, fertilizers, antibiotics, and algicides. The system also includes cooling sections in thermal contact with the headspace, a mixing device, various measurement devices, and temperature control equipment. The interior of the upper chamber may be processed with additives, coatings, or physical modifications to optimize light scattering distribution. The shape of the chamber can vary and include various forms such as tubular, circular, rectangular, etc. Materials used for the chamber construction include plastics with UV-blocking agents, wavelength-optimizing coatings, anti-fouling coatings, and structures to maintain the chamber's structural integrity. Ethanol is collected in the tray at a concentration higher than the growth medium to ensure the quality of the final fuel. This system is designed with the goal of sustainably and efficiently producing ethanol using genetically enhanced microorganisms.

A US invention with U.S. Pat. No. 8,372,613B2 which was granted on Feb. 12, 2013 titled “Methods and compositions for ethanol producing cyanobacteria” relates to a transgenic Synechocystis cyanobacterium host cell containing a synthetic nucleic acid construct. This construct includes a light-responsive promoter (psbAII), sequences that encode the enzymes pyruvate decarboxylase (pdc) and alcohol dehydrogenase (adh), and a transcription termination sequence. The construct can be introduced into the host cell as an expression vector, such as the pMota plasmid, or integrated into the cell's chromosome. The transgenic cyanobacterium, whether wild-type or the NDH-2(−) mutant strain of Synechocystis sp. PCC 6803, uses the psbAII promoter along with the pdc and adh sequences derived from the Zymomonas mobilis pLOI295 plasmid to produce ethanol in recoverable amounts of over 10 millimoles after approximately five days of cultivation, with some cases reaching up to about 13 millimoles. These cyanobacteria, under light-stimulating conditions with light intensities of approximately 1000 μE m−2s−1, produce ethanol that can be harvested and utilized as a biofuel. Additionally, the method includes genetic modifications to increase ethanol production capacity in cyanobacteria, which can contribute to the development of renewable energy sources and reduce reliance on fossil fuels.

A US invention with U.S. Pat. No. 8,895,272B2 which was granted on Nov. 25, 2014 titled “Methods for the economical production of biofuel from biomass” relates to a method for producing distiller's dried grains from a fermentation process for the production of isobutanol, which includes the following steps: First, a genetically engineered yeast biocatalyst is cultured in a fermentation chamber containing a growth medium with at least one carbon source, such as six-carbon sugars (glucose, galactose, mannose) or five-carbon sugars (arabinose, xylose), to produce isobutanol. This genetically engineered yeast contains foreign genes encoding enzymes such as acetolactate synthase, keto acid reductoisomerase, dihydroxy acid dehydratase, keto-isovalerate decarboxylase, and isobutyraldehyde dehydrogenase. After isobutanol production, at least part of the insoluble material, including the yeast biocatalyst, is separated from the fermentation chamber, and by drying it, distiller's dried grains are obtained. Additionally, the soluble residues from the fermentation process may be added to the dried grains to produce a combination of grains and soluble materials, which includes unconsumed solids, nutrients, proteins, fibers, and oils. This method also allows the use of dried corn as a carbon source, which involves steps such as grinding, forming a corn slurry, and contacting it with alpha-amylase and glucoamylase enzymes to produce a corn liquid. The primary goal of this invention is to produce isobutanol along with high-quality dried grains while reducing raw material waste through the recycling and reuse of fermentation residues.

A US invention with U.S. Pat. No. 8,152,867B2 which was granted on Apr. 10, 2012 titled “Process, plant and biofuel for integrated biofuel production” relates to an integrated method for producing butanol and biodiesel from biomass, which includes the following steps: First, the removal of hexose from feedstocks such as sugarcane, corn, or sugar beets to form lignocellulosic materials is carried out. Then, hexose is converted into butanol through fermentation. After that, the lignocellulosic materials are split into pentose and residues, with the pentose being converted into biodiesel via microbial or algal processes. The residues are used for energy production, for example, by burning them in a heater or boiler. Additionally, the biodiesel produced reacts with alkyl materials such as methanol, yielding biodiesel and glycerin, where the glycerin can be further converted into additional biodiesel. The mass ratio of biodiesel to butanol in this process is at least about 0.2 kiloton of biodiesel to 1.0 kiloton of butanol. This method allows the use of various carbon sources, including both five-carbon and six-carbon sugars, and optimizes the utilization of various biomasses such as bagasse, reed, and agricultural waste. The primary goal of this invention is to simultaneously produce two types of biofuels with high efficiency and to reduce raw material waste through recycling processes and the conversion of by-products.

A US invention with U.S. Pat. No. 8,895,279B2 which was granted on Nov. 25, 2014 titled “Applications of the rotating photobioreactor” relates to an integrated process for removing and recovering nutrients from a nutrient-rich liquid input stream using autotrophic or phototrophic microorganisms in a rotating photobioreactor. This system includes a chamber with a headspace, a transparent upper section for sunlight entry, and a lower section containing an aqueous growth medium with cultures of enhanced microorganisms such as algae and cyanobacteria. The process includes the following steps: First, light is applied to the microorganisms to enable photosynthesis. Then, a carbon source is introduced into the chamber, and the liquid input stream is passed along growth plates, which are partially submerged in the liquid. In this setup, the microorganisms remove nutrients from the input stream, absorb them into the biomass, produce oxygen, and the output liquid stream becomes nutrient-depleted. Additionally, the system is equipped with means to harvest the microorganisms from the growth plate surfaces without diluting them into the output stream. Other features of this invention include the type of chamber coating, materials used for the growth plates, continuous or intermittent harvesting methods, and precise control of environmental conditions such as pH and temperature. Furthermore, the process may involve the use of strategic gases such as air, methane, nitrogen, and oxygen to optimize microorganism performance and harvest by-products like ammonia and water. The primary goal of this invention is to improve the efficiency of nutrient removal from liquid input streams and produce biomass that can be utilized for organic fertilizer production or energy generation.

A US invention with U.S. Pat. No. 9,150,888B2 which was granted on Oct. 6, 2015 titled “Engineered CO2 fixing microorganisms producing carbon-based products of interest” relates to engineered photosynthetic microorganisms for biofuel production, specifically a Synechocystis cyanobacterium host cell. This microorganism is designed by inserting an artificial nucleic acid construct that includes a light-responsive promoter (psbAII promoter), sequences encoding the enzymes pyruvate decarboxylase (pdc), NADPH-dependent alcohol dehydrogenase (adh), and catalase. The enzymes used are derived from plasmids of Zymomonas mobilis and Moorella sp. HUC22-1, and the psbAII promoter is derived from Synechocystis sp. PCC 6803. This engineered cyanobacterium, whether wild-type or the NDH-2(−) mutant strain, is capable of producing ethanol in recoverable amounts greater than 10 mmol within five days under appropriate light conditions. The goal of this genetic engineering is to enhance ethanol production as a sustainable biofuel and reduce dependence on fossil fuels. Additionally, these microorganisms are designed without the need for additional DNA markers, allowing for optimized and harvestable ethanol production.

A US invention with U.S. Pat. No. 9,284,579B2 which was granted on Mar. 15, 2016 titled “Ethanol production in microorganisms” relates to engineered photosynthetic microorganisms for ethanol production, involving the introduction of recombinant nucleic acid sequences to encode the enzymes pyruvate decarboxylase (pdc) and alcohol dehydrogenase (adh). In this method, the copy number of the alcohol dehydrogenase sequence in the engineered microorganisms is higher than the copy number in control microorganisms, which have a single adh gene and a single pdc gene under one promoter. This genetic arrangement enables the engineered microorganisms to produce higher ethanol yields compared to the non-engineered microorganisms under the same cultivation conditions. Furthermore, to optimize enzyme expression, various promoters are used, including inducible and constitutive promoters, with the copy numbers of the pdc and adh genes being controlled by chemically regulated promoters. These microorganisms are primarily cyanobacteria, and in some cases, include the removal of competing genes, such as lactate dehydrogenase, to ensure optimized and harvestable ethanol production. The main objective of this genetic engineering is to enhance ethanol production as a sustainable biofuel and reduce dependence on fossil fuels.

A US invention with U.S. Pat. No. 10,519,471B2 which was granted on Dec. 31, 2019 titled “Organisms for photobiological butanol production from carbon dioxide and water” relates to a photobiological method for butanol production, comprising the following steps: First, a genetically engineered photosynthetic microorganism, containing recombinant nucleic acid sequences that encode the enzymes pyruvate decarboxylase (pdc) and alcohol dehydrogenase (adh), is cultivated in a medium under light and inorganic carbon conditions. These microorganisms are capable of converting Calvin cycle intermediates, such as glyceraldehyde 3-phosphate, 3-phosphoglycerate, fructose-1,6-bisphosphate, and fructose-6-phosphate, into butanol using the added enzymes. The energy and reducing power required for butanol synthesis are provided through photosynthetic water splitting and electron transfer processes with a proton gradient. Next, the produced butanol is harvested through an isolation process. This method, especially when using microorganisms such as Chlamydomonas reinhardtii or Synechococcus elongatus, is capable of producing significantly higher amounts of butanol compared to non-engineered microorganisms under similar cultivation conditions. The main goal of this method is to enhance butanol production as a sustainable biofuel and reduce dependence on fossil fuels.

A US invention with U.S. Pat. No. 11,781,162B2 which was granted on Oct. 10, 2013 tiled “Two-stage process for producing oil from microalgae” relates to an integrated method for biofuel production from algae, comprising the following steps: First, the production of oil-producing algal biomass is achieved through photoautotrophic growth using light and inorganic carbon. Then, by employing a stress-induction mechanism, the heterotrophic growth of oil-producing algae is initiated, where sugar is added as the primary carbon and energy source to stimulate algal oil production. Finally, algal oil is extracted from the algae using mechanical methods such as cell wall disruption, or biological methods involving the use of biological agents to break down the cell wall or the walls of oil vesicles. This method may also include the conversion of the extracted oil into biodiesel. Additionally, the process may involve adding nitrogen-fixing algae, such as cyanobacteria, to supply nitrogen as a nutrient. Various types of algae, including diatoms, green algae, cyanobacteria, and other hybrid species, are utilized in this method. The primary objective of this invention is to improve the efficiency of biofuel production from algae by combining photoautotrophic and heterotrophic growth, optimizing the use of carbon and energy resources, and effectively extracting algal oil for the production of sustainable biofuels.

A US invention with publication No. US20210355071A1 which was filed on Aug. 15, 2007 titled “Enhanced production of fatty acid derivatives” relates to a process for producing fatty acid derivatives, such as fatty alcohols, by genetically engineered cells. These cells contain exogenous genes that encode enzymes, including thiolase, Acyl-CoA synthetase, Acyl-CoA reductase, and alcohol dehydrogenase, which utilize the fatty acid biosynthesis pathway. The cells are specifically designed to produce biofuels, including biodiesel, fatty alcohols, and fatty acid esters. The process involves cultivating recombinant cells in a medium containing a carbon source and extracting biofuel products, such as esters or fatty alcohols, while minimizing environmental impacts and optimizing the use of biological resources. This process also has the potential to reduce costs and increase efficiency in biofuel production.

A US invention with U.S. Pat. No. RE48,308E1 which was granted on Nov. 17, 2020 titled “Processes for producing fuels and biofertilizers from biomass and products produced” relates to a process for converting biomass into liquid fuels, chemical feedstocks, and cyanobacteria-based biofertilizers. The process involves the following steps: first, biomass is converted into hydrocarbon liquids through hydroprocessing. These liquids are then upgraded to produce liquid fuels and chemicals. Next, structured biochar with pore sizes ranging from 20 to 400 angstroms is produced through microwave pyrolysis of biomass residues. Additionally, the carbon dioxide generated in these steps is used to cultivate cyanobacteria in a photobioreactor. Finally, a biofertilizer containing structured biochar and cyanobacteria is produced. This fertilizer can be enhanced by adding diazotrophic microorganisms or nutrients absorbed into the biochar. Furthermore, structured biochar is used as a moisture retention agent to promote the growth of agricultural crops such as soybeans. The primary goal of this invention is to optimize the production of biofuels and sustainable fertilizers for agriculture.

The large-scale production of carbon dioxide by industrial plants is a major factor in climate change and air pollution. This invention aims to reduce these greenhouse gases and convert them into biofuels using microorganisms. The present invention, focused on reducing the effects of greenhouse gases and utilizing carbon dioxide from industrial smokestacks to produce biofuels from cyanobacteria, represents a significant departure from previous technologies. It emphasizes the absorption and recovery of CO₂ from industrial gases using amine solvents (such as monoethanolamine) and transferring it to cyanobacteria cultivation systems.

### DESCRIPTION OF THE INVENTION

The present invention relates to a process for producing biofuels through the use of microorganisms, particularly cyanobacteria, and carbon dioxide extracted from industrial chimneys. One of the main objectives of this invention is to reduce the negative effects of greenhouse gases and to convert surplus industrial carbon dioxide into renewable energy sources. In this method, carbon dioxide is extracted from the flue gases of industrial chimneys using capture and disposal systems, and then transferred to an environment where cyanobacteria are cultivated. In this environment, the cyanobacteria use photosynthesis to produce biofuel.

The first step involves separating and purifying carbon dioxide (CO₂) from the exhaust gases of industrial chimneys so that it can be used in the cultivation process of cyanobacteria. For this purpose, a gas capture and disposal system, consisting of an absorption tower and a disposal tower, is utilized.

The absorption tower (FIG. 2, No. 201) captures CO₂ from the exhaust gas stream using an absorbing solvent. The exhaust gases from the chimney enter the bottom of the absorption tower, while the absorbing solvent flows downward from the top of the tower. This countercurrent flow enhances the contact surface between the gas and the liquid, leading to efficient CO₂ absorption. The solvent used in this invention is an amine-based compound, particularly monoethanolamine (MEA), due to its high absorption capacity and suitable reactivity with CO₂. In this stage, the temperature is maintained between 40 to 60 degrees Celsius to achieve maximum absorption efficiency.

The CO₂-rich solution exiting the absorption towers is then transferred to the disposal tower (FIG. 2, No. 202). In this tower, heat is applied to separate the CO₂ from the solvent, thereby regenerating the solvent. The applied heat (typically between 100 to 120 degrees Celsius) causes the CO₂ to detach from the solvent and exit as a gas from the top of the disposal tower. The regenerated solvent is returned to the absorption cycle, which helps reduce costs and increase the sustainability of the process. The CO₂ gas exiting the disposal tower may contain small amounts of water vapor and other impurities.

The CO₂ gas exiting the disposal tower is first cooled to allow the condensation and separation of water vapor. In the next stage, gaseous impurities such as nitrogen, oxygen, and hydrogen sulfide are removed using separation membranes, resulting in pure CO₂. The purified CO₂ gas is then compressed using appropriate compressors. Finally, the compressed CO₂ is stored in pressure vessels (FIG. 3) and transferred to the cultivation environment through corrosion-resistant piping, while control and monitoring systems continuously track the purity, pressure, and temperature of the gas to ensure safe and optimal use in the cyanobacteria cultivation process.

The pure and compressed carbon dioxide is introduced into a shallow, large-area pool (FIG. 4) via piping. This pool has insulated walls and is covered with a glass or polycarbonate cover (FIG. 4, No. 401) to prevent contamination while allowing sunlight to effectively reach the cultivation environment. CO₂ is distributed in the cultivation environment in the form of fine bubbles through microbubble diffusers (FIG. 4, No. 403). This distribution enhances the solubility and availability of CO₂ to the cyanobacteria. The CO₂ transfer piping is made from PVC or polypropylene pipes, which are resistant to corrosion caused by CO₂. Diffusers are placed at regular intervals on the pool floor to ensure uniform distribution of CO₂.

The depth of the pool is typically set between 20 to 30 centimeters to maximize light penetration, ensuring that all cyanobacteria can benefit from sunlight. The surface area of the pool varies depending on the desired production capacity and can range from several hundred square meters to several thousand square meters. The walls and floor of the pool are constructed using insulating and corrosion-resistant materials such as high-density polyethylene (HDPE), polypropylene (PP), or fiberglass reinforced with durable resins. The floor of the pool is designed with a gentle slope toward a collection point, facilitating easier biomass harvesting and water drainage.

To optimize the growth of cyanobacteria, aeration and mechanical stirring are applied within the pool. Aeration, through the injection of air or CO₂ from the pool's floor, not only supplies the necessary oxygen but also creates circulation and mixing, preventing cell sedimentation. Mechanical mixers or air currents help evenly distribute nutrients and gases throughout the cultivation environment, ensuring that all cells receive uniform exposure to light. At night, LED lights are used to continue the photosynthesis process and prevent the growth of cyanobacteria from stopping.

Temperature, pH, and nutrient sensors are installed at various points within the pool and are connected to a central control system. This system continuously monitors vital parameters to maintain optimal conditions for cyanobacteria growth. The pH of the cultivation environment is maintained between 6.5 and 8.5, and the temperature is kept between 20 and 30 degrees Celsius to provide the ideal conditions for cyanobacteria. Nutrient chambers (FIG. 4, No. 402) located beside the pool supply the necessary nutrients, which are injected into the cultivation environment through pumps when the system detects the need. Additionally, antibacterial materials compatible with cyanobacteria are used to prevent the growth of unwanted microorganisms and contaminants.

In one embodiment, the cyanobacteria used is a strain of Synechocystis sp. PCC 6803, selected for its high efficiency in photosynthesis and biofuel production.

In addition, the installation of an inlet filtration system for air, water, and injected gases such as CO₂ and air is crucial. By using appropriate filters, including HEPA filters for air and mechanical and microbiological filters for water, the entry of suspended particles, unwanted microorganisms, and contaminants into the system can be prevented. These measures, along with regular maintenance and replacement of filters and monitoring their performance, help maintain the health of the culture, improve the efficiency of the process, and prevent equipment failure.

During the harvesting stage, the cyanobacteria biomass is separated from the cultivation medium using a centrifuge. First, the culture medium containing the cyanobacteria is collected from the cultivation pool and transferred to the centrifuge unit (FIG. 5, No. 502). In the centrifuge, the sample is placed in rotating rotors and spun at high speeds. The centrifugal force generated causes the cyanobacteria cells, which have a higher density than the cultivation medium, to move toward the outer walls of the rotor, where they accumulate. Meanwhile, the clear liquid remains in the center.

After the centrifugation process is completed, the biomass that has accumulated at the bottom of the rotor or centrifuge container is collected. This biomass is then harvested and prepared for the next stages. The remaining clear liquid can either be returned to the cultivation system if needed or disposed of properly (FIG. 5, No. 501).

The centrifugation process can process large volumes of culture medium in a short amount of time and also results in nearly complete separation of the cells from the culture medium, leading to higher harvesting efficiency. The collected biomass has a high concentration, which facilitates the subsequent drying stage.

The next stage is drying. In the drying stage, the cyanobacteria biomass is dried using a spray drying method (FIG. 5, No. 503). In this process, the liquid biomass, after being separated from the culture medium, is sprayed into a drying chamber where warm air with controlled temperature flows. The fine particles of biomass come into contact with the hot air, quickly losing their moisture and turning into dry powder. The use of a spray dryer also allows for precise temperature control, which prevents the thermal degradation of sensitive compounds in the biomass.

In the next step, the dried cyanobacteria biomass is ground using a ball mill (FIG. 6, No. 602). In this process, the biomass is placed into a rotating cylinder containing metal or ceramic balls. As the cylinder rotates, the balls collide with the biomass, breaking it down into fine and uniform particles. Grinding with the ball mill increases the surface area of the biomass, which facilitates the extraction of active compounds, such as lipids, in subsequent stages. This mechanical grinding optimizes the preparation of the biomass for further processes such as extraction and conversion into biofuel.

The next stage involves lipid extraction from cyanobacteria using solvents. The dried and ground biomass is placed into an extraction chamber (FIG. 6, No. 601), typically a tank or reactor made of stainless steel resistant to corrosion. This chamber is equipped with a mechanical stirring system (FIG. 7) and temperature control to maintain optimal conditions for extraction. The biomass is mixed with a suitable organic solvent, such as hexane, methanol, ethanol, or a combination of these. The choice of solvent is based on the polarity of the lipids and the efficiency of extraction. The solvent-to-biomass ratio is usually between 5:1 and 10:1 to ensure complete contact between the solvent and the cells.

The extraction process is carried out under controlled conditions. The temperature of the chamber is typically set between 30 and 60° C. to enhance the solubility of the lipids without damaging their structure. Continuous stirring ensures that the solvent effectively penetrates the biomass and extracts the lipids from the cell walls. The extraction time can range from 1 to 4 hours, depending on the desired efficiency and the type of solvent used. After extraction is complete, the mixture is separated by filtration or centrifugation (FIG. 8, No. 802) to isolate the solid residue from the lipid-containing liquid. The solvent containing the lipids is then transferred to an evaporation unit, where the solvent is recovered, and the purified lipids are prepared for the next steps in biofuel production.

The next step is the transesterification of the extracted lipids from the cyanobacteria to convert them into biofuels such as biodiesel. This process takes place in a chemical reactor (FIG. 8, No. 801), typically made of stainless steel to resist heat and corrosion. The lipids are mixed with a short-chain alcohol, such as methanol or ethanol, in the presence of a catalyst (usually sodium hydroxide or potassium hydroxide). The reaction is carried out at a temperature between 50 and 60° C. with continuous stirring for 1 to 2 hours. These conditions are optimal for breaking down triglyceride molecules and converting them into methyl or ethyl esters (biodiesel) and glycerol.

In the biodiesel-glycerol separation stage, after the transesterification process is completed, the reaction mixture is transferred to a settling tank or decanter (FIG. 9). Due to differences in density and polarity, biodiesel (methyl or ethyl esters of fatty acids), being lighter and less polar, settles in the upper layer, while glycerol, due to its higher density and polar nature, collects in the lower layer. This separation typically occurs under the force of gravity without the need for complex equipment, and it may take between 1 to 8 hours for the layers to fully separate. To expedite this process and achieve more effective separation, centrifugation can be used, applying centrifugal force to speed up the layering process. Once the two distinct layers have formed, biodiesel is carefully removed from the upper part of the tank, while glycerol is collected from the lower section. This step is critical as any remaining glycerol in the biodiesel could negatively affect the fuel quality, leading to issues such as filter blockages and reduced engine efficiency.

The characteristics of the produced biodiesel include an appropriate cetane number, high flash point, and oxidative stability, which make it an efficient and environmentally friendly biofuel. This biodiesel can be used as a replacement or additive for fossil diesel fuels.

During the transesterification process, in addition to producing biodiesel, glycerol is also obtained as a by-product. Glycerol is a trihydroxy alcohol with moisturizing and softening properties, making it valuable in various industries. It is particularly used in the production of soaps and personal care products such as creams, lotions, shampoos, and toothpaste. The use of glycerol derived from the biodiesel production process not only helps reduce production costs but also contributes to sustainable development and the reduction of industrial waste by utilizing a valuable by-product.

In the lipid extraction process from cyanobacteria using solvents, after the lipids are separated, the used solvents are recovered and recycled through distillation. The solvent-lipid mixture is transferred to a distillation unit, where the solvent is evaporated by increasing the temperature under vacuum and then condensed back into a liquid in a cooling condenser. The recovered solvent, after purification, is reused in the extraction process, which helps reduce costs and minimize chemical waste. Additionally, the remaining culture medium after biomass harvesting can be returned to the cultivation system after filtration and sterilization, with the addition of necessary nutrients. This reuse of the culture medium saves water and nutrient resources, reducing the environmental impact of the process while contributing to the overall sustainability and economic efficiency of the system.

### BRIEF DESCRIPTION OF FIGURES

FIG. 1 shows overview of the invention.

FIG. 2 shows a view of the absorption tower and disposal tower, including:
201: Absorption tower
202: Disposal tower

FIG. 3 shows a view of the carbon dioxide storage pressure vessels.

FIG. 4 shows a view of the cultivation pool and nutrient chambers, including:
401: Glass or polycarbonate cover
402: Nutrient chambers
403: Microbubble diffusers

FIG. 5 shows a view of the centrifuge unit and spray dryer, including:
501: Water return pipe to the pool
502: Centrifuge unit
503: Spray dryer

FIG. 6 shows a view of the ball mill and lipid extraction unit, including:
601: Lipid extraction unit
602: Ball mill

FIG. 7 shows a view of the lipid extraction unit and mechanical stirring system.

FIG. 8 shows a view of the centrifuge unit and transesterification unit, including:
801: Transesterification unit
802: Centrifuge unit

FIG. 9 shows a view of the settling tank.

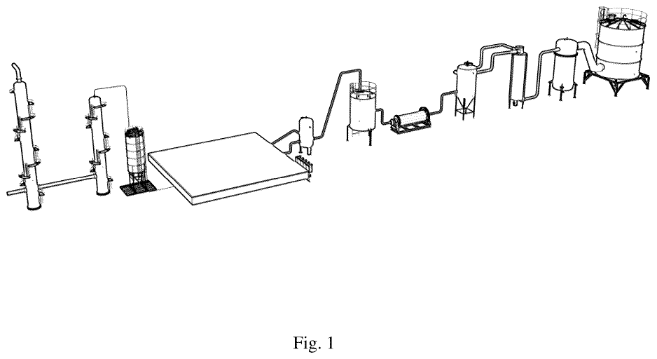

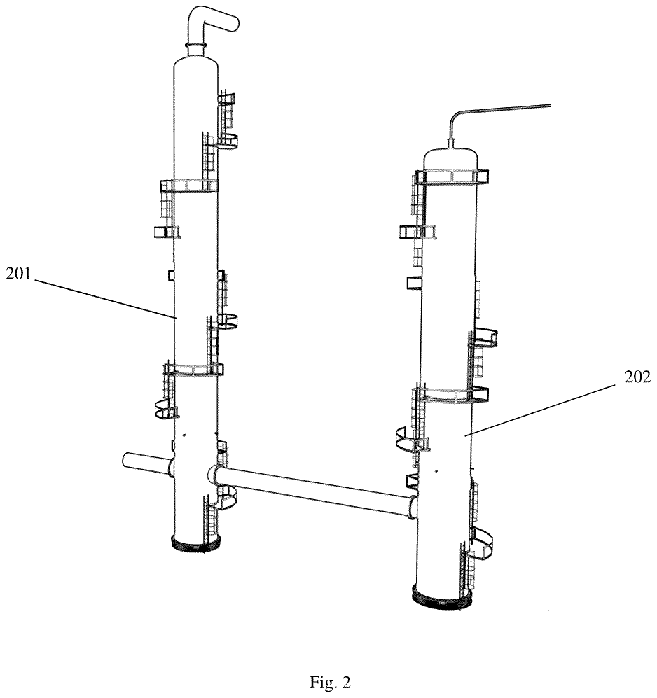

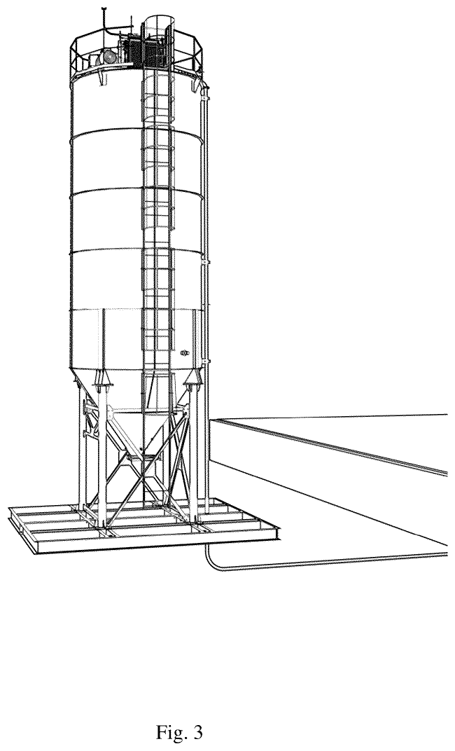

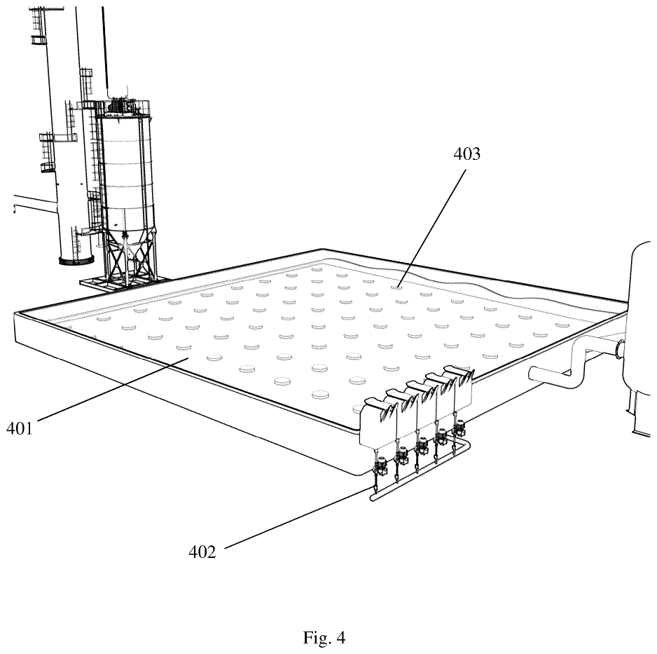

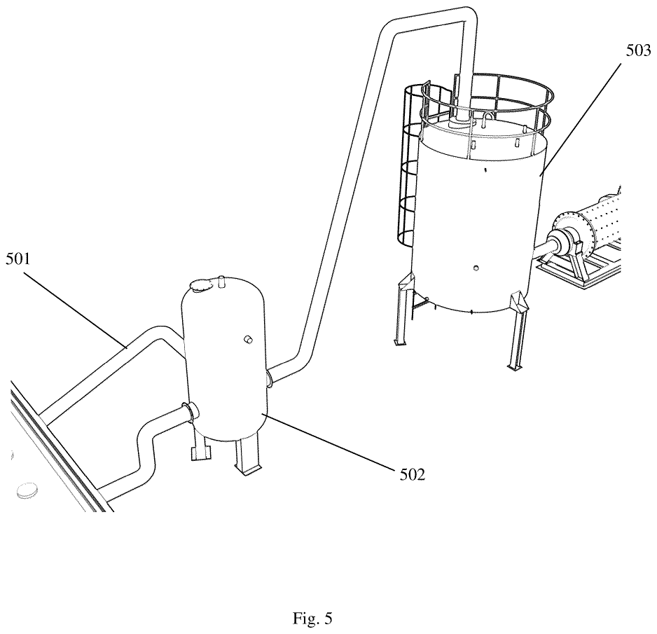

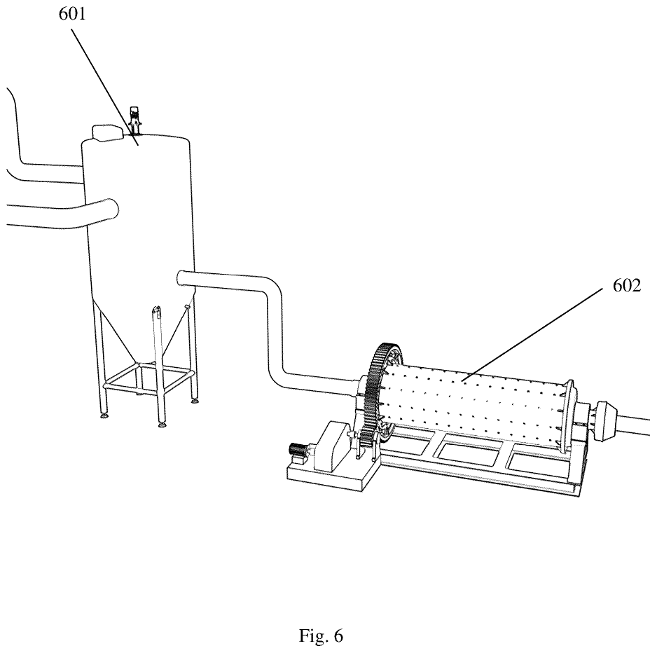

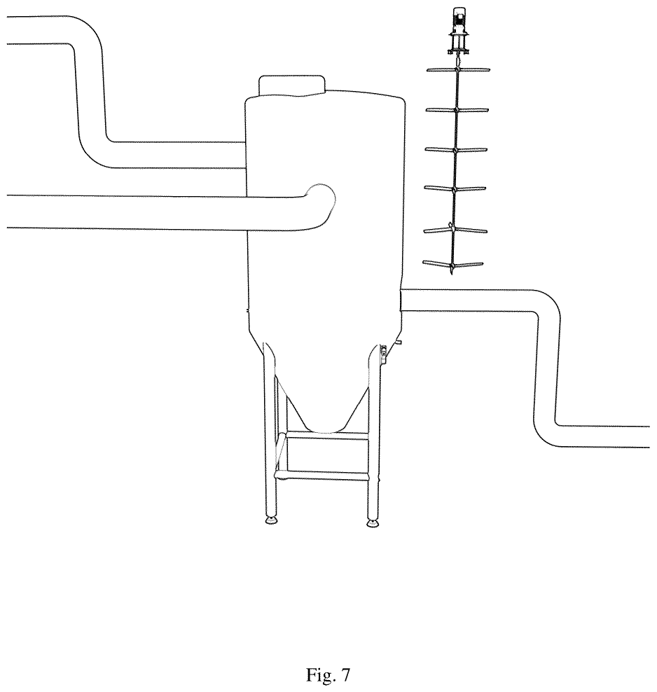

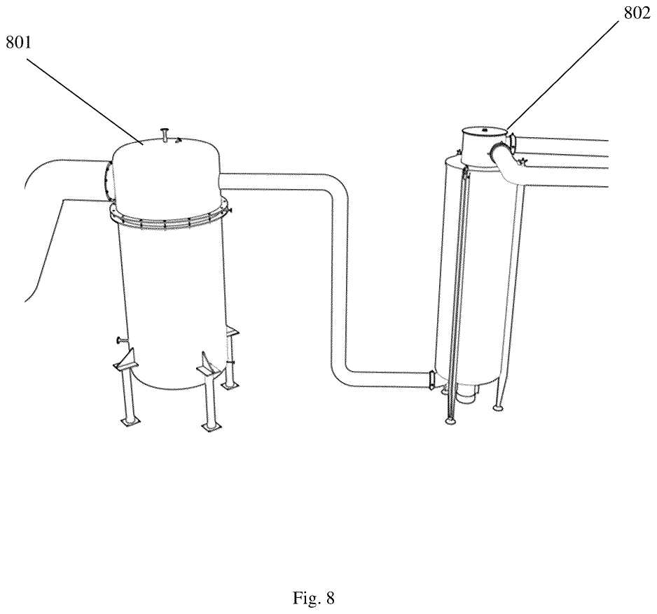

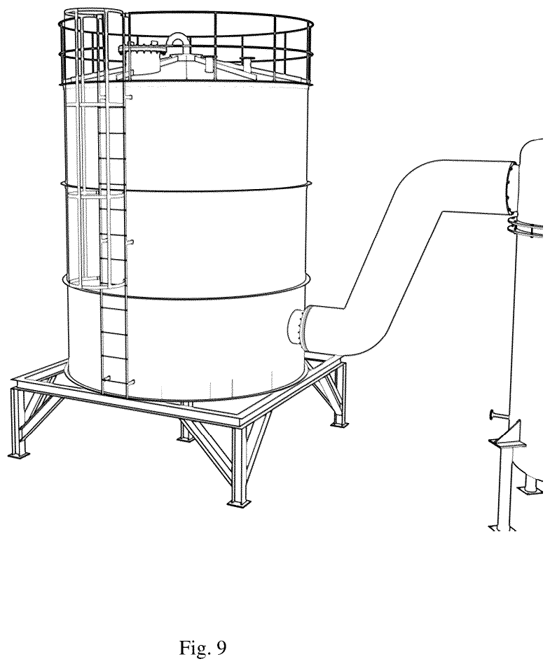

What is claimed is:

1. The invention of a process of producing biofuel using microorganisms and carbon dioxide extracted from industrial chimneys, which includes at least one system for separating and purifying carbon dioxide from the exhaust gases of industrial chimneys, including at least one absorption tower and at least one disposal tower and at least one system for transferring pure and compressed carbon dioxide to the cyanobacteria cultivation environment, including at least one corrosion-resistant pipeline and at least one cyanobacteria cultivation environment, including at least one shallow pool with insulated walls and a glass or polycarbonate coating and at least several microbubble diffusers, and at least one aeration system and at least one mechanical stirrer and at least one control and monitoring system including temperature sensors, pH sensors, and nutrient sensors, and a central control system and at least one cyanobacteria biomass harvesting unit, including at least one centrifuge and at least one lipid extraction unit from the biomass, including at least one spray dryer and at least one ball mill and at least one unit for converting the extracted lipids into biofuel, including at least one transesterification reactor and at least one used solvent recovery system and at least one biodiesel separation unit from glycerol, including at least one sedimentation tank.

2. The biofuel production process according to claim 1, wherein carbon dioxide absorption is carried out using an amine solvent, specifically monoethanolamine, at a temperature between 40 to 60 degrees Celsius in the absorption tower.

3. The biofuel production process according to claim 1, which includes solvent recovery in the disposal tower by applying heat at a temperature of 100 to 120 degrees Celsius.

4. The biofuel production process according to claim 1, wherein the purified carbon dioxide is stored under pressure in tanks, and the control and monitoring system oversees purity, pressure, and temperature.

5. The biofuel production process according to claim 1, wherein the cultivation pool has insulated walls and a glass or polycarbonate coating to prevent contamination and enhance sunlight penetration.

6. The biofuel production process according to claim 1, wherein microbubble diffusers are arranged at regular intervals on the floor of the pool for the uniform distribution of carbon dioxide.

7. The biofuel production process according to claim 1, wherein the pool has a depth between 20 to 30 centimeters and its walls and floor are made of durable materials such as high-density polyethylene (HDPE).

8. The biofuel production process according to claim 1, which includes aeration and mechanical stirring of the pool to prevent cell sedimentation and ensure uniform distribution of nutrients.

9. The biofuel production process according to claim 1, wherein the cyanobacteria biomass is separated using a centrifuge and the remaining clear liquid is returned to the system.

10. The biofuel production process according to claim 1, wherein the separated biomass is subsequently dried using a spray dryer at controlled temperatures to prevent the degradation of sensitive materials.

11. The biofuel production process according to claim 1, wherein the dried biomass is ground into fine particles using a ball mill.

12. The biofuel production process according to claim 1, wherein lipid extraction from the biomass is performed using solvents such as hexane, methanol, or ethanol.

13. The biofuel production process according to claim 1, wherein the solvent after lipid extraction is recovered via distillation and reused in the process.

14. The biofuel production process according to claim 1, wherein the extracted lipids from cyanobacteria are converted into biofuel such as biodiesel using a transesterification process with alcohols such as methanol and catalysts such as sodium hydroxide.

15. The biofuel production process according to claim 1, wherein the biodiesel produced in the transesterification process is separated from glycerol using gravitational force or centrifugation.

16. The biofuel production process according to claim 1, wherein the separated glycerol is recovered as a by-product for the production of soaps and cosmetic and personal care products.
